(19) United States
(12) Patent Application Publication
(10) Pub. No.: US 2020/0401904 A1
(43) Pub. Date: Dec. 24, 2020

(54) ADAPTIVE MEDICAL IMAGING DEVICE CONFIGURATION USING ARTIFICIAL INTELLIGENCE

(71) Applicant: GE Precision Healthcare LLC, Wauwatosa, WI (US)

(72) Inventors: Anurag Voleti, Waukesha, WI (US); Sridhar Nuthi, Waukesha, WI (US)

(21) Appl. No.: 16/450,480

(22) Filed: Jun. 24, 2019

(51) Int. Cl. G06N 3/12; G06N 20/00; G06N 3/08

(52) U.S. Cl. CPC G06N 3/126; A61B 8/58; G06N 3/086; G06N 20/00

(57) ABSTRACT

Methods, apparatus, systems and articles of manufacture to provide a mutatable machine genetic structure are disclosed. An example apparatus includes memory including instructions for execution by a processor and a machine genetic structure specifying composition, performance, and health of a machine; and at least one processor. The processor is to execute the instructions to at least: evaluate the machine genetic structure with respect to an operating condition of the machine to identify a discrepancy and/or an opportunity for improvement for the machine genetic structure to satisfy the operating condition; determine a mutation of the machine genetic structure from a first sequence to a second sequence to address the discrepancy and/or opportunity for improvement to satisfy the operating condition; and set the machine genetic structure from the first sequence to the mutation of the second sequence to configure the machine for operation according to the machine genetic structure.

### FIELD OF THE DISCLOSURE

This disclosure relates generally to medical systems, and, more particularly, to adaptive medical system configuration using artificial intelligence.

### BACKGROUND

Manufacturers of large machines (e.g., imaging machines in health care, turbines in energy, and engines in transportation) deploys such large machines to users/customers for use in the field. Due to the complications of such machines, some manufactures provide repair and/or upkeep services with teams of technicians to service the machines during scheduled maintenance and/or when the machine is malfunctioning and/or down. When a user has a problem with a deployed machine, the user contacts the manufacturer (e.g., via call, email, etc.) describing the problem (e.g., providing symptoms) and a technician is sent to fix the machine. Additionally, the manufacture and/or customer can schedule maintenance calls at set durations of time to verify that the machine is working properly

### BRIEF SUMMARY

Certain examples provide an apparatus including memory including instructions for execution by at least one processor and a machine genetic structure specifying composition, performance, and health of a machine; and at least one processor. The at least one processor is to execute the instructions to at least: evaluate the machine genetic structure with respect to an operating condition of the machine to identify at least one of a discrepancy or an opportunity for improvement for the machine genetic structure to satisfy the operating condition; determine a mutation of the machine genetic structure from a first sequence to a second sequence to address the at least one of a discrepancy or an opportunity for improvement to satisfy the operating condition; and set the machine genetic structure from the first sequence to the mutation of the second sequence to configure the machine for operation according to the machine genetic structure.

Certain examples provide a non-transitory computer readable storage medium including instructions. The instructions, when executed, cause a machine to at least: evaluate a machine genetic structure with respect to an operating condition of the machine to identify at least one of a discrepancy or an opportunity for improvement for the machine genetic structure to satisfy the operating condition, the machine genetic structure specifying composition, performance, and health of the machine; determine a mutation of the machine genetic structure from a first sequence to a second sequence to address the at least one of a discrepancy or an opportunity for improvement to satisfy the operating condition; and set the machine genetic structure from the first sequence to the mutation of the second sequence to configure the machine for operation according to the machine genetic structure.

Certain examples provide a method including evaluating, by executing an instruction using at least one processor, a machine genetic structure with respect to an operating condition of the machine to identify at least one of a discrepancy or an opportunity for improvement for the machine genetic structure to satisfy the operating condition, the machine genetic structure specifying composition, performance, and health of the machine. The example method includes determining, by executing an instruction using the at least one processor, a mutation of the machine genetic structure from a first sequence to a second sequence to address the at least one of a discrepancy or an opportunity for improvement to satisfy the operating condition. The example method includes setting, by executing an instruction using the at least one processor, the machine genetic structure from the first sequence to the mutation of the second sequence to configure the machine for operation according to the machine genetic structure.

### BRIEF DESCRIPTION OF THE DRAWINGS

FIG. 1 illustrates an example medical machine configuration system or apparatus in communication with one or more medical machines.

FIG. 2 illustrates an example implementation of the machine configuration processor of the example of FIG. 1.

FIG. 3 shows an example machine genetic structure representing an image quality of an imaging scanner.

FIG. 4 depicts an example illustration of a machine genetic structure including a plurality of mutations to modify at least one of the composition, performance, or health of its corresponding machine.

FIG. 5 illustrates an example imaging system function to gene mapping.

FIG. 6 provides another illustration of an example genetic algorithm to drive a machine genetic sequence for an imaging system.

FIG. 7 illustrates an example table showing an operating condition, machine genetic structure, and fitness assessment score associated with the genetic structure for the operating condition.

FIG. 8 shows an example table providing a performance score from scoring a particular gene for a plurality of operating conditions.

FIG. 9 illustrates an example mutation or intervention to adjust configuration of a machine.

FIG. 10 depicts changes in genetic structure at design time, at run time, and during down time.

FIG. 11 illustrates an example in which the genetic structure of a machine breaks down due to a failure.

FIGS. 12-13 are flowcharts representative of machine readable instructions which can be executed to evaluate and modify machine genetic structure using the example system of FIGS. 1-2.

FIG. 14 is a block diagram of an example processing platform structured to execute the instructions of FIGS. 12-13 to implement the system of FIGS. 1-10.

FIG. 15 is a block diagram of an example processing platform that can form part of a medical machine including a machine genetic structure according to the example system of FIG. 1.

The figures are not to scale. In general, the same reference numbers will be used throughout the drawing(s) and accompanying written description to refer to the same or like parts.

The features and technical aspects of the system and method disclosed herein will become apparent in the following Detailed Description set forth below when taken in conjunction with the drawings in which like reference numerals indicate identical or functionally similar elements.

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

As used herein, the terms “system,” “unit,” “module,” “engine,” etc., may include a hardware and/or software system that operates to perform one or more functions. For example, a module, unit, or system may include a computer processor, controller, and/or other logic-based device that performs operations based on instructions stored on a tangible and non-transitory computer readable storage medium, such as a computer memory. Alternatively, a module, unit, engine, or system may include a hard-wired device that performs operations based on hard-wired logic of the device. Various modules, units, engines, and/or systems shown in the attached figures may represent the hardware that operates based on software or hardwired instructions, the software that directs hardware to perform the operations, or a combination thereof.

Descriptors “first,” “second,” “third,” etc. are used herein when identifying multiple elements or components which may be referred to separately. Unless otherwise specified or understood based on their context of use, such descriptors are not intended to impute any meaning of priority, physical order or arrangement in a list, or ordering in time but are merely used as labels for referring to multiple elements or components separately for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for ease of referencing multiple elements or components.

### I. Overview

Fleets of machines, such as, but not limited to, imaging systems, turbines and engines are increasingly being deployed over large geographic regions. In the medical field, imaging systems including modalities such as magnetic resonance imaging (MRI), computed tomography (CT), nuclear imaging, and ultrasound are increasingly being deployed in hospitals, clinics, and medical research institutions for medical imaging of subjects. Engines deployed in locomotives or aircrafts, need to operate under varying environmental conditions. In power generation systems, wind turbines or water turbines are installed to harvest energy from natural resources. For facilities owning a machine belonging to a fleet of machines, it is desirable to maximize utilization of the machine with minimal downtime. However, system failures and breakdowns interrupt the workflow processes involving the machine and reduce its utilization.

Most manufacturers strive to provide effective periodic maintenance routines and responsive or on call repair services. Despite the refined capability of preventive maintenance programs, machines can sometimes develop problems which need out of turn diagnosis and repair. Usually, such problems are identified by a concerned authority at the facilities that manage the installed machine. The identified problems are submitted as service requests in one or more formats, such as, but not limited to, a textual description through a webform and a voice call through a helpline. As used herein, the term “service request” refers to a description of a problem, a fault, or issue associated with a machine, such as an imaging system. The problem, fault, or issue can be observed during routine maintenance check, or during usage of the machine, for example, by a technician or a user. The service request can be a description in text or audio message provided by the user via a user interface and can be automatically stored in a database.

Traditionally, servicing of a machine among the fleet of machines such as the imaging systems can require parts replacement or on-site visits by a field engineer to the site. Such on-site visits by field engineers can be expensive and time consuming for both customer and system manufacturer or repair facility, who typically arranges for such visits. Remote diagnosis and repair are often used to expedite system repair and obviate or minimize the need for such on-site visits. However, existing remote diagnosis and repair still entails the need to interrupt use of the imaging system and contact with the repair facility. Also, upon identification of a fault using remote diagnosis, manual intervention can be needed to submit a service request, initiate service request processing, and identify the requirement of an on-field visit. Traditionally, an expert is required to manually scan huge amount of data pertaining to service requests, to make and/or recommend decisions about servicing options based on the service requests. Manual processing of service requests is inefficient and adversely effects the response time. Reducing manual overhead while processing servicing requests without compromising on accuracy and response times is desirable.

Examples disclosed herein provide systems and methods that characterize and enhance a machine, such as an imaging system, etc., by defining a machine configuration as a genome and facilitating configuration, modification, and operation of the machine by modifying that genome (referring to herein as a “MuGene”, for example). The MuGene represents a machine's configuration, status/health, etc., which can be read, modified, processed, analyzed, and/or otherwise used for machine configuration, operation, fleet analysis, etc.

In certain examples, a machine's MuGene is an executable construct in which a performance of the machine, its health, its longevity, its endurance, etc., can be enhanced using machine learning and/or self-learning algorithms provided via a cloud-based platform to learn from a greater population of assets of a same family while contextualizing the performance of that specific asset through intervention in contextual operating conditions of the specific asset. When applied in context of a medical equipment manufacturing field such as MRI/CT systems, an example use case includes an intervention through specific code snippets that, like a human genetic make-up, can drive quality of the output, such as improved image quality in sub-optimal conditions, etc. Other example use cases include driving an increased number of exams per incident, zero unplanned down-time, etc., thereby improving throughput and, eventually, a lower total cost of ownership of assets. The same concept can be applied to a diverse set of industry portfolios in which the performance of an asset is critical to drive entitlement of investments, for example.

In certain examples, device intercommunication and/or interconnectivity, such as the Internet of Things (IoT), enables enhanced interaction with and between imaging systems and/or other devices including human-computer interaction, human-machine interaction, machine-machine interaction, machine-cloud interaction, etc. Certain examples enhance such interactions with machine-driven intelligence to not only enhance and automate the decision-making skills of humans using data but also enable a machine to adapt to contextual reality of its operating environment.

Certain examples define a machine gene or MuGene as a collection of nano-, micro-, and macro-level parameters, settings, descriptors, etc., such as material composition (molecular level), manufacturing process, machine assembly, configuration, operating conditions, hardware and software, etc. Every individual machine in a family of similar machines is unique due to intricate and inherent differences resulting from a core composition, manufacturing process, and/or other aspects of the machine gene. Within a given machine, the concept of a machine gene can be extended to each individual component and sub-component of a machine including an ultimate child component that cannot be further disintegrated/decomposed, for example.

A collection of genes that form a machine as an individual entity also make that machine unique in terms of how it performs, how it responds to usage and operating conditions, its ability to heal and recover from problems, etc. This uniqueness that differentiates one machine from every other machine within the same product family is represented as a machine gene. The machine gene can be leveraged to determine what makes one machine better than other machines in a product family for a given operating criterion. In certain examples, one or more aspects of a machine gene can be mutated and/or enhanced to improve machine performance. As machines and material design evolve, the mutational aspect can be driven by the machine itself in response to a changing context such that a given machine is always at its peak performance, for example.

Certain examples enable a machine or system to compensate for one system's weakness with another system's capability to address an operational use case, even at a sub-optimal level. For example, based on machine gene processing and configuration, a low-resolution scanned image can be obtained from a computed tomography (CT) scanner in low power conditions, and/or better image reconstruction algorithms can be used to compensate for poor image data capture during a scan.

Certain examples drive improved imaging machine configuration, operation, performance, etc., through creation, manipulation, and management of machine genes. In certain examples, composition of a machine gene is identified, and the machine gene is analyzed with respect to its ecosystem (e.g., a fleet of machines and their associated machine genes, etc.). Model(s) are built to capture machine genetic characteristics with respect to one or more ecosystems, operating conditions, etc. Design of Experiments (DOE) and simulations are used to identify a combination of machine genetic characteristics that works best against a given ecosystem, operating condition, etc. A framework is defined to collect and analyze data to define and refine the machine genetic characteristics with respect to the ecosystem, operating condition, etc. Machine genetic characteristic(s) can be mutated, enhanced, and/or otherwise modified to configure machine component(s) (e.g., hardware, software, firmware, etc.) to respond to the ecosystem, operating condition, etc. In certain examples, one machine component can compensate for another machine component as part of a mutation of the machine gene sequence.

More specifically, machine genetic composition can be identified by identifying one or more nano, micro, and/or macro factors that influence specific aspects of a machine and its components. For example, machine form, function, capability, and/or other characteristic can be specified by one or more factor(s). The factors provide a combination of hardware, software, process, manufacturing, and materials, for example, that influence the machine's form, function, capability, other characteristic(s), etc. In manufacturing, two machines coming out of the same assembly line may not be the same due to variation induced from how individual materials are composed, cast, processed, connected, and assembled, etc. By analyzing factors inducing variation between machines along with other data points taken for DOE, etc., the combination of factors influencing a capability of the machine such as scanning, detecting, moving, vibrating, cooling, etc., forms the core of the machine gene (MuGene). Continuous analysis of a fleet of machines over a time period helps improve the composition of each machine gene.

The machine gene can then be analyzed against its ecosystem. For example, a machine's genetic structures can be compared against a fleet of machine genes. For example, advanced statistical analysis can be executed with respect to a fleet of machines to identify which combination of factors would make a given machine the most optimal machine configuration with respect to the ecosystem and operating conditions surrounding the machine. Unconstrained and randomized sample sets can be analyzed using various statistical techniques to identify a combination and composition of machine genes that identify a given outcome as bad, good, or excellent, for example.

In certain examples, models can be built to capture (e.g., continuously, periodically, on demand, etc.) the genetic characteristics for comparison with respect to one or more ecosystems, operating conditions, etc. Correlation and causation of multi-variate generic characteristics can be identified to make a specific gene better than other configurations for a given ecosystem and operating condition, for example.

Using DOE and simulations, a combination of genetic characteristics can be determined to work best against a given ecosystem and/or operating condition, for example. The combination may be different from a current genetic composition of a given machine or component, for example. An ability to determine an appropriate combination of genetic characteristics against different simulations of ecosystem and/or operating conditions helps drive design tolerances and flexibility of hardware and/or software aspects of a machine (e.g., an imaging machine, diagnostic device, etc.), for example. A framework can be defined to collect and analyze data from a machine to define and refine the genetic characteristics of the machine with respect to one or more ecosystems and/or operating conditions, for example.

In certain examples, a genetic characteristic or a combination of characteristics related to a machine or it's component (e.g., software, firmware, and/or hardware) can be mutated and/or enhanced. Such mutation/enhancement can initially be a reactive intervention, for example, that can be incrementally expanded to proactive, preventative, and/or predictive intervention as applicable generic characteristic(s) are locked down and solidified for a given ecosystem and/or environment condition(s), for example.

As part of the continuous learning and analysis, engineering and technology design alternatives can be integrated to compensate for one component(s)' capability with other component(s) that are already included in the machine built and/or added as part of the mutation to compensate for failure(s) of a given component. An ability to understand the design mitigations that are built-in to a machine and/or can be added to the machine (e.g., through a software update, new hardware accessory, etc.) increases a likelihood that a component overcompensates when another component of a machine enters a failure mode. However, the same capability may also help the machine enter a failsafe mode rather than a catastrophic failure mode for the entire machine and/or one or more machine components, for example.

A mutational gene is a gene that compensates for an under-performing feature gene or a sub-optimal performing gene by changing the conditions under which such a gene is performing to rectify the impact of those anomalies in those genes. A performance enhancing MuGene combines different strands of a genetic composition in association with a given system and its functionality to improve performance given one or more operating conditions, usage variations, etc.

Specific genes can be recognized in each machine product family through data analytics, machine/deep learning, etc., and can be correlated with product capability(-ies). A product capability can be formed as a collection of these genes coming together to perform a specific operation. For example, an ability of a computed tomography (CT) scanner to scan a patient can be linked to various genetic underpinnings such as radiation dose, high voltage, detector fidelity, reconstruction algorithm(s), stability of gantry, noise avoidance, etc. Certain examples first determine how these genes individually adjust to a changing operating context and, then, collectively compensate to derive an expected outcome utilizing machine learning and collective memory.

Certain examples identify, characterize, and/or classify a machine and/or individual component(s) of the machine as features associated with genes forming a machine genome for the device. Characteristics driving machine performance, machine behavior, etc., in different condition(s) can be determined using the genes, which drives improved diagnosis, trouble shooting, and prescriptive mitigation/repair, for example. Additionally, the machine gene can mutate to drive incremental changes and adoptions to automatically help the machine to compensate itself against specific failure mode(s)/condition(s), etc.

Traditional approaches to machine troubleshooting and adjustment often waste material and result in complex mitigations through part replacement and design changes. Further, in most cases, such a traditional approach is not an effective solution to the problem. Additionally, traditional approaches take a binary view of a machine status as working or not working. The lack of a self-compensating design or mitigation often leads to an only option of solving the problem with direct service intervention and replacing the problematic component.

Another challenge that is often ripe in a traditional approach is correlation of failure signatures and component capabilities to data at a higher abstraction, which does not take into consideration how the machine is actually manufactured, what materials are used, how the machine is assembled, etc. In contrast, certain examples determine nano, micro, and macro characteristics to define a machine gene and provide very precise and cost-effective interventions to adjust a machine and also lead to better design of a machine and its components.

Thus, certain examples provide a machine (e.g., an imaging device, medical device, health information system, etc.) and/or a computer, processor, and/or other device configuring the machine to prescribe specific machine genes and enhancement offerings based on a target customer install base. Machine genes can be integrated with asset performance management (APM) to provide very prescriptive asset performance offerings, for example. Advanced systems can be designed and tied to specific gene advancement algorithms, for example. In certain examples, self-learning, self-healing, and self-improving can be provided in an imaging scanner and/or other machine using machine genes processed using deep learning, other machine learning, and/or other machine cognition, for example.

Using machine genes, a machine and its components can be modeled and evaluated individually and in combination, with their own characteristics and inherent capacities, all connected at the genetic level. The machine genes enable precise description and control of a machine's state and performance, for example. The “MuGene” provides a deep modeling and understanding of the physics and intricate design of the particular machine, connected with operational and usage context. By integrating the knowledge of the MuGene with deep learning and/or other machine intelligence algorithms, machine configuration and operation can be modeled, predicted, configured, improved, repaired, etc.

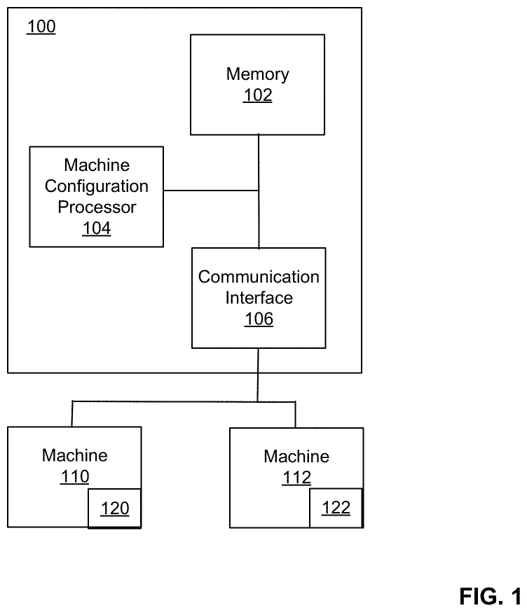

FIG. 1 illustrates an example medical machine configuration system or apparatus 100 in communication with one or more machines 110, 112 (e.g., an imaging scanner, medical device, medical information system, etc.). Each machine 110-112 includes a machine genome or MuGene 120-122 defining the configuration of its respective machines 110-112. The one or more machine genes 110-112 define structure, configuration, operation, status, etc., for the respective machine 110, 112. The example machine configuration apparatus 100 includes memory 102, a machine configuration processor 104, and a communication interface 106. The example machine configuration apparatus 100 communicates with the machines 110-112 via the communication interface 106 (e.g., a wireless and/or wired interface, etc.) to extract information regarding the machine's genetic code 120-122, adjust and/or otherwise configure the code 120-122, etc.

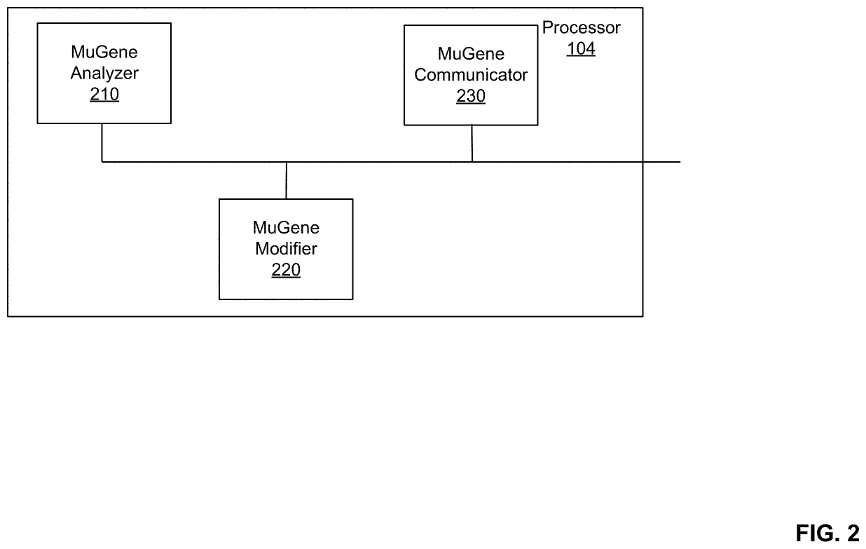

FIG. 2 illustrates an example implementation of the machine configuration processor 104 of the example of FIG. 1. As shown in the example of FIG. 2, the machine configuration processor 104 can be implemented to include a MuGene analyzer 210, a MuGene modifier 220, and a MuGene communicator 230.

The example MuGene analyzer 210 processes the MuGene 120-122 information received from the machine 110-112 via the communication interface 106 to determine the machine's 110-112 configuration, status, error, capability, etc. The MuGene analyzer 210 can determine whether the machine 110-112 is able to handle a particular task, is configured properly for a given workflow/task/operation, is operating without fault, etc.

In certain examples, the MuGene 120-122 is an executable function such as software code that contextualizes software to adapt to operating conditions based on a population of machines 110-112 and a view of such a fleet of machines 110-112 with respect to an individual machine 110-112. The MuGene 120-122 is a self-learning algorithm that enhances the genetic make-up or configuration of the machine 110-112. For example, the MuGene 120-122 takes a population view from cloud to edge to contextualize software and machine operating settings to adapt to the machine's operating conditions and a target on which the particular machine 110-112 is being operated.

Thus, the MuGene 120-122 takes a global fleet and environment view to focus on particular hardware, firmware, and software components of a particular machine 110-112 and how the hardware, firmware, and software elements of the machine 110-112 interact with internal and external conditions of the machine 110-112 and its environment, for example. The MuGene 120-122 can be defined, for example, for an outcome, Y, as follows:

Outcome(Y)=function(Hardware, Software, Operating Conditions(Parameters, Environment, Operated on, Others) (Eq. 1),

taking into account the particular asset, the cloud-based environment of multiple assets, and the edge device/connectivity between the individual asset and the cloud, for example.

The MuGene analyzer 210 can determine composition genetics (e.g., manufacture, composition/makeup, variance against tolerance, software, etc.) for the machine 110-112, performance genetics (e.g., performance of the MuGene 120-122 under specific operating conditions, etc.) for the machine 110-112, and health genetics (e.g., composition and performance to classify health of different outputs, a boundary or threshold or limitation on machine health, etc.) for the machine 110-112 through analysis of the MuGene 120-122. Mutation of all or part of the MuGene 120-122, such as by the machine 110-112 and/or the MuGene modifier 220, adjusts the composition, performance, and/or health of the corresponding machine 110-112 based on best practices and/or settings from another machine 110-112, observations/ground truths associated with a workflow or task, user specification, healthcare protocol, etc. The MuGene communicator 230 can communicate with the machine 110-112 to extract its MuGene 120-122 and/or update/replace the machine's MuGene 120-122 with an updated/replacement MuGene 120-122 after analysis/processing, for example.

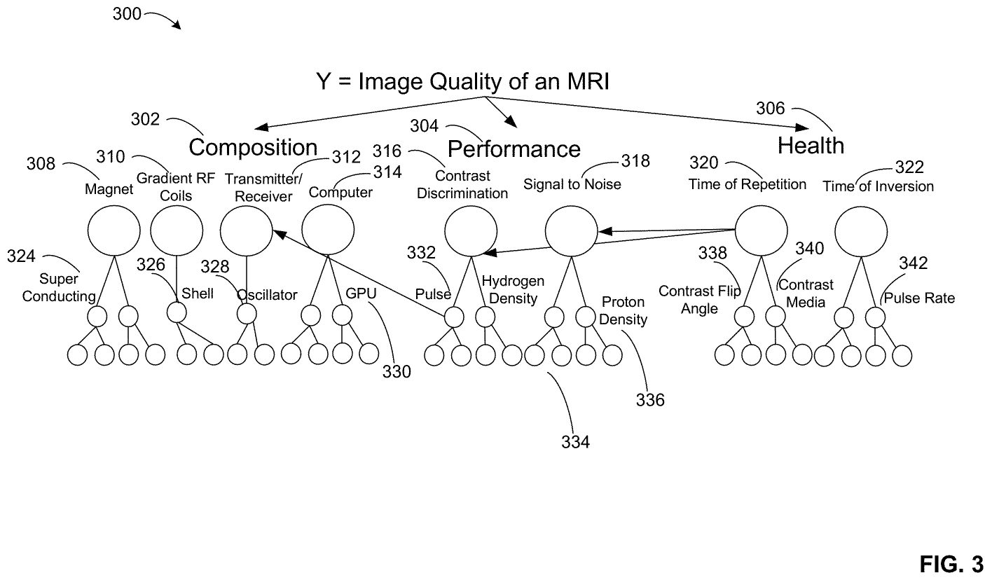

FIG. 3 shows an example MuGene Y 300 representing an image quality of an MRI scanner. The example genome Y 300 includes a segment related to composition 302, a segment related to performance 304, and a segment related to health 306. In the example of FIG. 3, the composition gene sequence 302 includes a magnet 308, gradient coils 310, radiofrequency (RF) transmitter/receiver 312, and a computer 314. As shown in the example of FIG. 3, the performance gene sequence 304 includes contrast discrimination 316 and signal to noise ratio 318. In the example of FIG. 3, the health gene sequence 306 includes a time of repetition 320 and a time of inversion 322. The time of repetition 320 is associated with the contrast discrimination 316 and the signal-to-noise ratio 318, for example. Those elements can be divided further, as shown in the example of FIG. 3.

For example, the magnet genome 308 can include a characterization/description of super-conducting properties 324 of the magnet 308. The gradient coil genome 310 can include a description of the coil shell 326, for example. The RF transmitter/receiver genome 312 can include a characterization of an included oscillator 328, for example. The computer genome 314 can include a description of the general processing unit (GPU) 330 associated with the computer 314, for example.

As shown in the example of FIG. 3, the contrast discrimination genome 316 can include a characterization/description of an associated pulse 332, which is also connected, as shown in the example of FIG. 3, to the RF transmitter/receiver 312. The signal-to-noise ratio genome 318 is further specified by a hydrogen density 334 and a proton density 336.

As shown in the example of FIG. 3, the time of repetition genome 320 can include a description of a contrast flip angle 338 and contrast media 340. The time of inversion genome 322 can include a pulse rate 342, for example.

In certain examples, a rank-based genetic algorithm can be used to combine individual machine genomes 120-122 for mutation into improved machine composition, performance, and health. For example, the rank-based genetic algorithm can be defined as follows:

ϕ(i)=κ·R(i) for i=1, . . . N (Eq. 2),

wherein i refers to an individual machine 110-112 and/or its MuGene 120-122, κ is a constant representing selective pressure, and its value is fixed between 1 and 2. Greater selective pressure values cause the fittest individual machines/machine characteristics to have more probability of recombination. The parameter R(i) represents a rank of individual i.

Using the rank-based genetic evaluation of Equation 2, a cross-over can be orchestrated from cloud to edge device to medical device 110-112 (e.g., imaging system, etc.) so that a best combination of genetic structure can be deployed for each asset 110-112. Mutation can be orchestrated from cloud to edge to device so that one gene can compensate for another gene's suboptimal performance. The algorithm of Equation 2, executed centrally by the MuGene analyzer 210 and/or locally by each machine 110-112, can provide a continuous process of improvement as the algorithm self-learns through orchestration between the cloud, the edge, and the asset 110-112, for example.

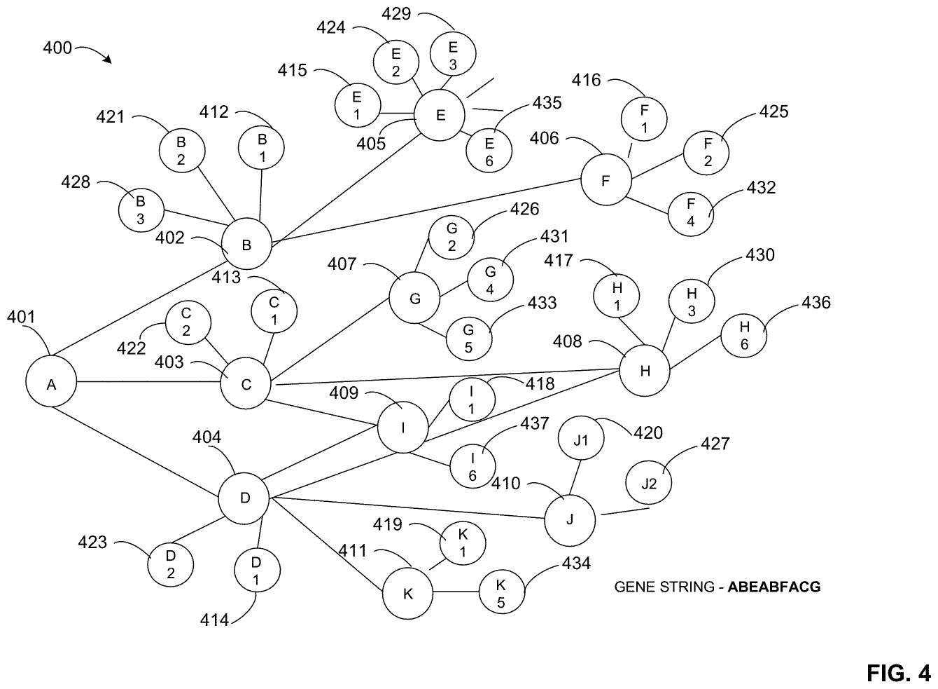

FIG. 4 depicts an example illustration of a machine MuGene 400 including a plurality of mutations to modify at least one of the composition, performance, or health of its corresponding machine 110-112. Genes A-K 401-411 represent a “standard”, normal, or preset configuration for the machine 110-112. As shown in the example of FIG. 4, many mutations can exist to adjust the configuration/operation of the machine 110-112 to suite a particular task, workflow, operating condition, error/failure, etc. For example, one or more genes 401-411 can have a first mutation 412-420. One or more genes 402-411 can have a second mutation 421-427, a third mutation 428-430, a fourth mutation 431-432, a fifth mutation 433-434, and/or a sixth mutation 435-437, for example. In the example of FIG. 4, the MuGene 400 can be formed according to a string or series of elements such as ABEABFACGACHACIADIADHADJADK, forming a picture or representation of the associated machine 110-112, its composition, performance/operation, and health/state, for example.

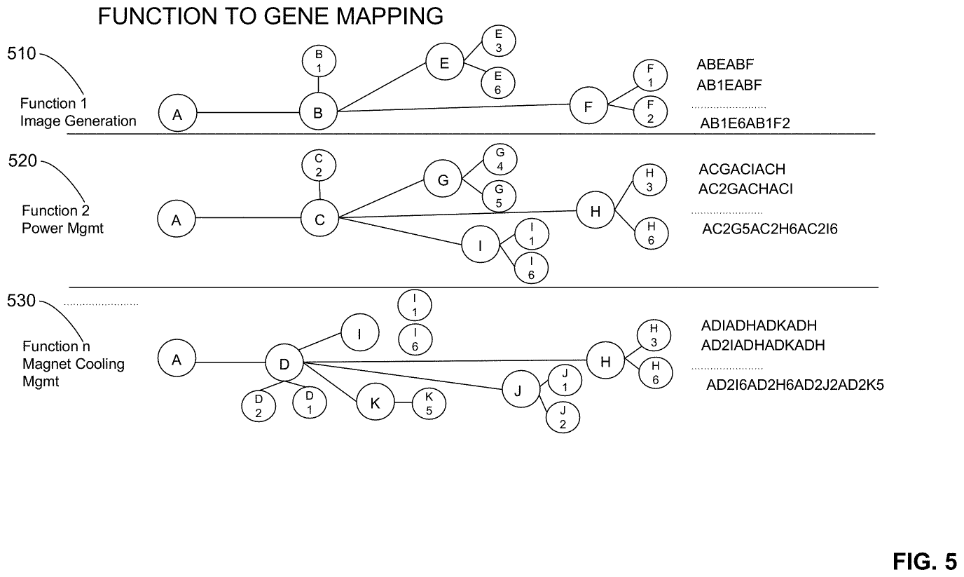

As shown in the example of FIG. 5, imaging system functions can be represented as gene mappings. As such, an adjustment to a function can take the form of a gene mutation (e.g., to adjust a time, an intensity, a focus, an arrangement, etc.), for example. The machine 110-112 executes according to the gene sequence (the MuGene 120-122) to operate according to its programmed code. FIG. 5 illustrates an example function to gene mapping for an image generation function 510, a power management function 520, and a magnet cooling function 530 for an MRI machine. As shown in the example of FIG. 5, each function 510-530 includes one or permutations/mutations/variants that can be dynamically selected/configured by the machine 110-112 and/or centrally by the machine configuration processor 104, for example. Thus, the machine 110-112 and/or the machine configuration processor 104 can adapt the machine to a particular task, operating condition, and/or other circumstance through selection of a genetic mutation for system configuration.

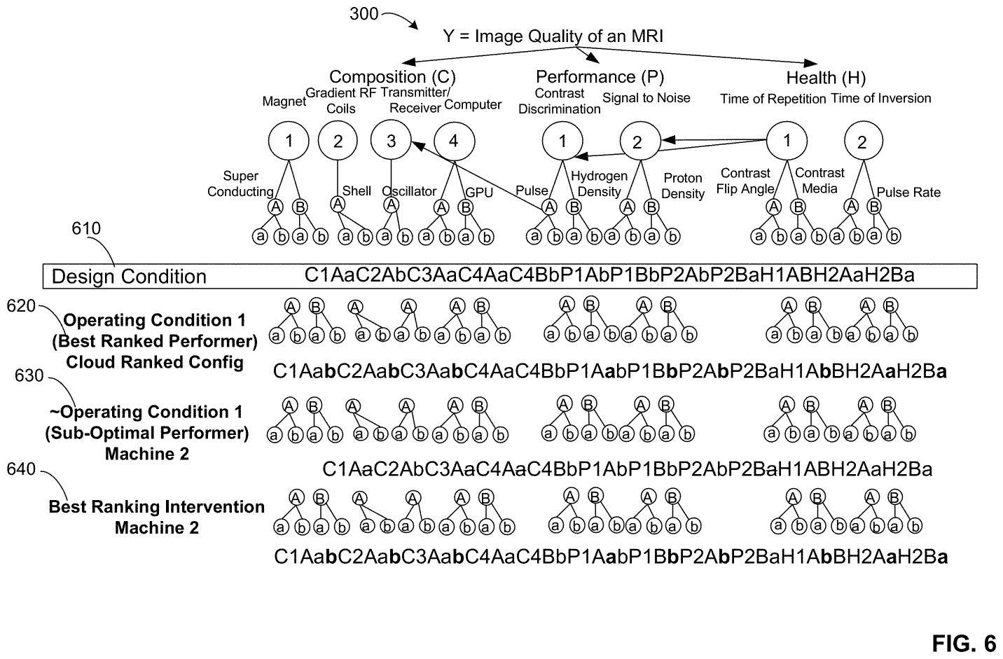

FIG. 6 provides another illustration of an example genetic algorithm to drive a machine genetic sequence, Y, for image quality in an MRI system. FIG. 6 expands on the example of FIG. 3 to take the genetic sequence 300 and a design condition 610 to evaluate gene sequence configurations/mutations according to a first operating condition. Example sequence 620 represents a genetic ranking of a best ranked performer among participating machines 110-112 organize in a cloud-based comparison (e.g., by the machine configuration processor 104, etc.) for the first operating condition. Example sequence 630 represents a genetic ranking of a sub-optimal performer among participating machines 110-112 organize in a cloud-based comparison (e.g., by the machine configuration processor 104, etc.) for the first operating condition. Example sequence 640 represents a genetic ranking of a best ranked intervention among participating machines 110-112 organize in a cloud-based comparison (e.g., by the machine configuration processor 104, etc.).

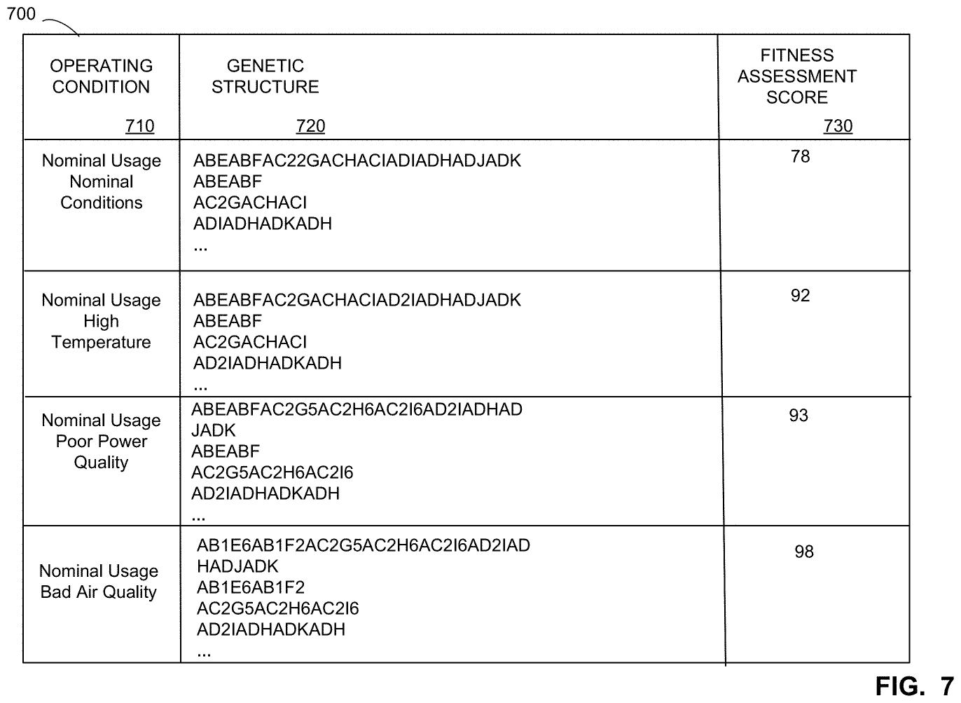

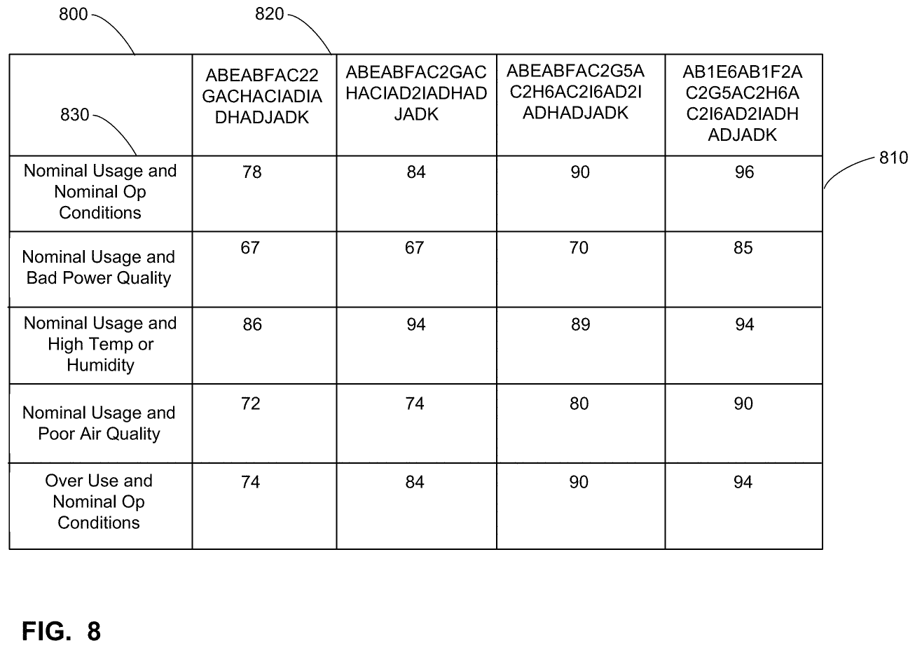

In addition to mapping functions to genes, such as in the example of FIG. 5, operating conditions can be mapped to genes as well, and gene performance with respect to mapped operating conditions can be determined. FIG. 7 illustrates an example table 700 showing operating condition 710, machine genetic structure 720, and fitness assessment score 730 associated with the genetic structure 720 for the operating condition 710. Thus, a particular gene can be scored (e.g., at a parent and component level, etc.) for a given operating condition. FIG. 8 shows an example table 800 providing a performance score 810 from scoring a particular gene 820 for a plurality of operating conditions 830. Based on the scoring 810, performance of a particular machine genetic structure 820 can be evaluated for a plurality of operating conditions 830 to derive best-in-class genetic structure baselines for each operating condition 830, for example. In certain examples, additional factors such as cost, complexity, time, customer expectation, benefit to effort analysis, etc., are considered in the determination of gene performance scores 820. Alternatively or in addition, such additional factors can be evaluated when operationalizing a gene mutation recommendation externally to one or more other machine(s) 110-112, for example.

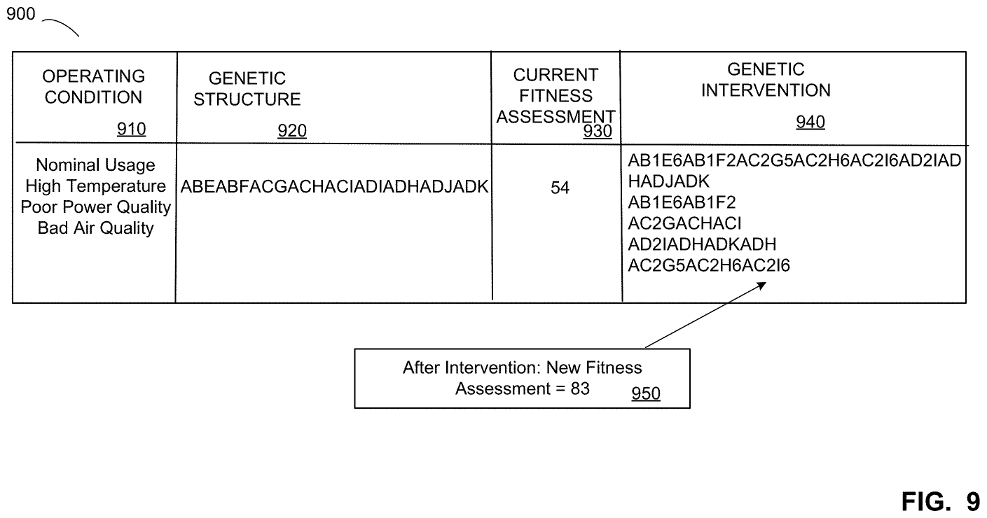

FIG. 9 illustrates an example mutation or intervention 900 to adjust configuration of a machine 110-112. As shown in the example 900 of FIG. 9, an operating condition 910 is specified along with a currently used, low performing genetic structure 920. A fitness assessment score 930 can be associated with the genetic structure 920, for example. A genetic intervention 940 can be provided to mutate and/or otherwise replace the low performing genetic structure 920, and an updated fitness assessment score 950 can be associated with the intervention 940, for example.

By modeling the genetic structure of each asset along with mapping of specific functions, capabilities, and operating conditions against a given parent or its sub-components, expected outcomes can be (continuously and/or periodically, etc.) monitored, measured, and analyzed with respect to actual outcomes through data science and analytics. As such, a specific asset can be analyzed to determine how it is performing against its current operating condition, an optimal genetic structure can be determined and recommended to address a current operating condition based on fleet analysis. This knowledge can be moved from cloud to edge to an actual machine 110-112 and its sub-components such that the intervention can be reactive, predictive, proactive, prescriptive, and personalized to specific customer expectations (e.g., performance, total cost of ownership, total cost of service, patient safety etc.), for example.

Gene compensation can happen at design time, at run time, and/or during down time as part of a service intervention, for example. As compensating interventions are captured and operationalized, the new genetic structure can be fitness scored at an overall parent level as well as at a subcomponent level along with how the new compensated system is interacting with its operating conditions. Advanced data science and analytics lead to new compensation opportunities by bringing in the data analysis to engineering design, for example.

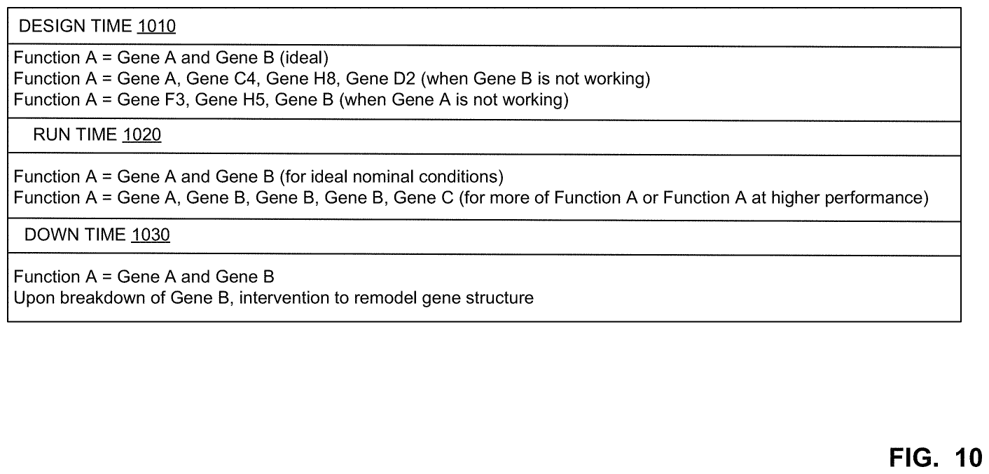

For example, FIG. 10 depicts changes in genetic structure at design time 1010, at run time 1020, and during down time 1030. In the example of FIG. 10, at design time 1010, function A is defined by a series of gene sequences 1012-1016. A first gene sequence 1012 is an “ideal” or desired or best practice configuration of the machine 110-112 to execute function A. A second gene sequence 1014 is an alternative configuration to be used when gene B is not working. A third gene sequence 1016 is an alternative configuration to be used when Gene A is not working.

In the example of FIG. 10, at run time 1020, function A is defined by another series of gene sequences 1022, 1024. A first gene sequence 1022 is ideal for nominal conditions. A second gene sequence 1024 is an alternative configuration to be used to execute more of function A and/or to execute function A by the machine 110-112 at a higher performance. For example, the machine 110-112 is configured to support more than a designed load, take more scans, etc., using the second gene sequence 1024 rather than the first gene sequence 1022 at run time 1020.

In the example of FIG. 10, in down time 1030, function A is defined by a gene sequence 1032 formed of Gene A and Gene B. However, in the example of FIG. 10, Gene B breaks down when a given threshold is crossed. If known compensation does not exist in a MuGene mutation, an intervention is executed to determine a new design, resulting in a remodeling to form a new gene structure. Scoring and fitness measurement can then be performed with respect to the new gene structure, for example.

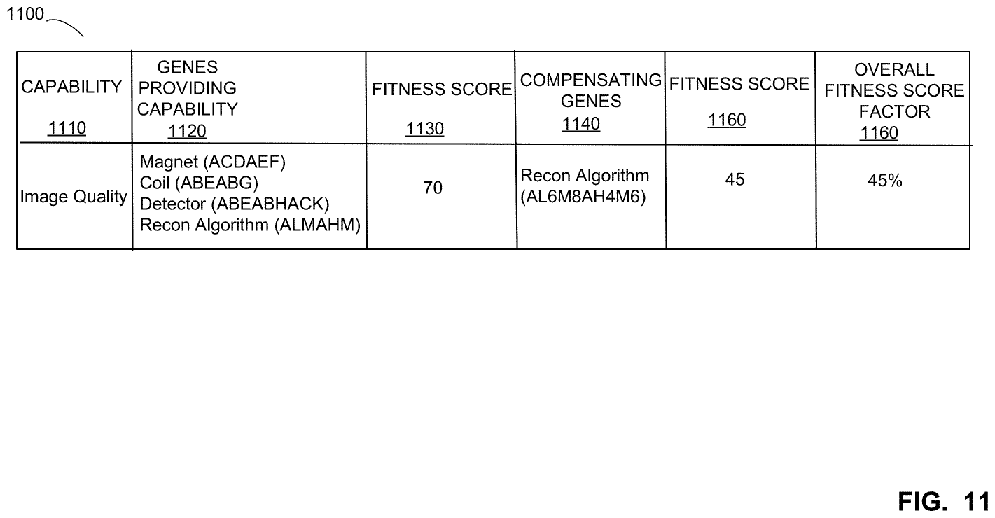

FIG. 11 illustrates an example 1100 in which the genetic structure of a machine 110-112 breaks down due to a software and/or hardware failure. For example, while the machine 110-112 assets appear to be intact, there is a breakdown of a particular capability. To compensate for the breakdown of that capability with other working component(s) and intact genetic structure(s), the machine 110-112 and/or the MuGene configuration processor 104 can maintain a table or other memory of possible compensation configurations such as shown in the example 1100 of FIG. 11. In the example of FIG. 11, an image quality capability 1110 is provided by a plurality of machine genes 1120 with an associated fitness score 1130 of genes 1120 to the capability/task 1110. However, in the example of FIG. 11, when a breakdown occurs in one or more components that support image quality of the MR system, poor image quality can result. A new reconstruction algorithm 1140 can be applied that is designed to address noise, curate errors, impute missing pixels, etc., to compensate for the breakdown in image quality. After compensation, a fitness score 1150 reflects the use of the reconstruction algorithm 1140 on the lower quality images, and an overall fitness score factor 1160 associated with the compensation.

Thus, the MuGene analyzer 210 can facilitate an analysis of operating condition(s), machine genetics, status, and available alternative(s) for one or more machines 110-112. The MuGene modifier 220 can facilitate mutation and/or replacement of the machine's MuGene 120-122 with another available gene sequence. The MuGene communicator 230 can receive MuGene 120-122 and/or other machine 110-112 information and can provide a MuGene 120-122 update and/or other configuration information to the machine(s) 110-112, for example.

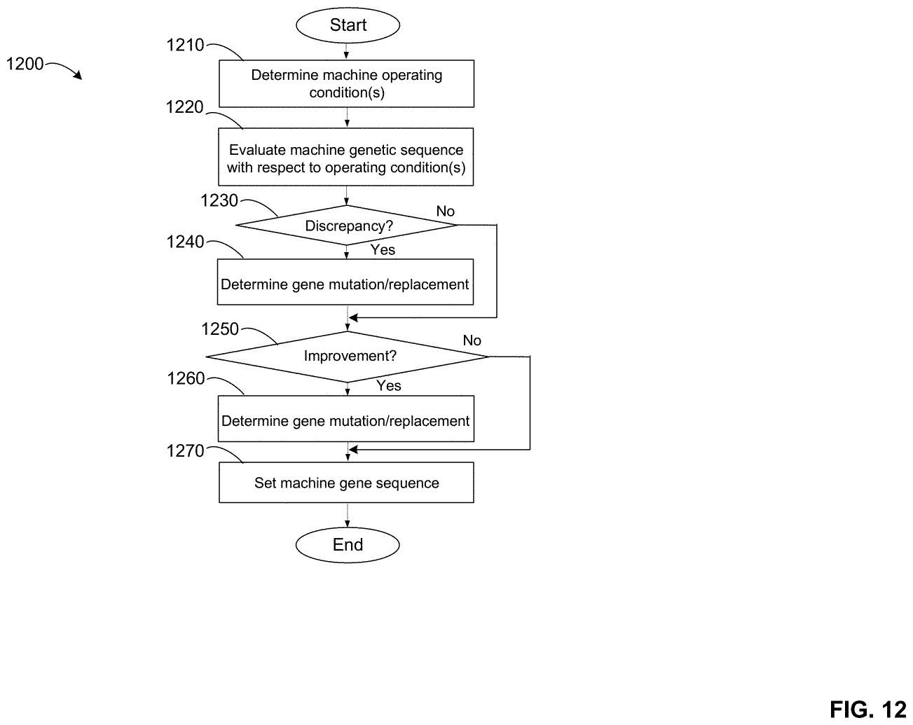

FIG. 12 illustrates a flow diagram of an example method 1200 to dynamically configure a machine 110-112 for operation according to one or more operating conditions. The example method 1200 can be formed from executable program instructions stored in memory and executable by at least one processor to implement the method 1200, for example. At block 1210, one or more operating conditions are determined for a machine 110-112. For example, one or more nano, micro, and/or macro factors influence specific aspects of the machine 110-112 and its components. For example, machine form, function, capability, and/or other characteristic can be specified by one or more factor(s). The factors provide a combination of hardware, software, process, manufacturing, and materials, for example, that influence the machine's form, function, capability, other characteristic(s), etc. In manufacturing, two machines 110-112 coming out of the same assembly line may not be the same due to variation induced from how individual materials are composed, cast, processed, connected, and assembled, etc. By analyzing factors inducing variation between machines 110-112 along with other data points taken for DOE, etc., the combination of factors influencing a capability of the machine 110-112 such as scanning, detecting, moving, vibrating, cooling, etc., forms the core of the machine gene (MuGene) 120-122. Continuous analysis of a fleet of machines 110-112 over a time period helps improve the composition of each machine gene 120-122, for example.

At block 1220, the genetic sequence 120-122 of the machine 110-112 is evaluated with respect to the operating condition(s). For example, a machine's genetic structures 120-122 can be compared against a fleet of machine genes 120-122. For example, advanced statistical analysis can be executed with respect to a fleet of machines 110-112 to identify which combination of factors would make a given machine 110-112 the most optimal machine configuration with respect to the ecosystem and operating conditions surrounding the machine 110-112. Unconstrained and randomized sample sets can be analyzed using various statistical techniques to identify a combination and composition of machine genes 120-122 that identify a given outcome as bad, good, or excellent, for example.

In certain examples, models can be built to capture (e.g., continuously, periodically, on demand, etc.) the genetic characteristics for comparison with respect to one or more ecosystems, operating conditions, etc. Correlation and causation of multi-variate generic characteristics can be identified to make a specific gene better than other configurations for a given ecosystem and operating condition, for example.

Using DOE and simulations, a combination of genetic characteristics can be determined to work best against a given ecosystem and/or operating condition, for example. The combination may be different from a current genetic composition 120-122 of a given machine or component 110-112, for example. An ability to determine an appropriate combination of genetic characteristics 120-122 against different simulations of ecosystem and/or operating conditions helps drive design tolerances and flexibility of hardware and/or software aspects of a machine 110-112 (e.g., an imaging machine, diagnostic device, etc.), for example. A framework can be defined to collect and analyze data from a machine 110-112 to define and refine the genetic characteristics 120-122 of the machine 110-112 with respect to one or more ecosystems and/or operating conditions, for example.

At block 1230, the evaluation is processed to determine whether an error, fault, and/or other discrepancy exists/has occurred with respect to the MuGene 120-122 and the operating conditions for the machine 110-112. For example, a discrepancy or disconnect between the machine's genetic configuration 120-122 and the operating condition(s) and/or other task at hand for the machine 110-112 is identified from the evaluation of gene sequence 120-122 with respect to operation condition(s). For example, the machine 110-112 may be missing a capability, a component may be malfunctioning, a configuration may be incorrect, etc., in comparison to machine operating condition(s) associated with the machine's ecosystem, environment, task, etc.

At block 1240, a mutation and/or replacement gene is determined to remedy/compensate for the error, failure, and/or other discrepancy between the current gene sequence 120-122 and the operating condition(s), task(s), etc., for the machine 110-112. For example, a genetic characteristic or a combination of characteristics 120-122 related to the machine 110-112 or its component (e.g., software, firmware, and/or hardware) can be mutated and/or enhanced. Such mutation/enhancement can initially be a reactive intervention (e.g., to an error, fault, other discrepancy, etc.), for example, that can be incrementally expanded to proactive, preventative, and/or predictive intervention as applicable generic characteristic(s) 120-122 are locked down and solidified for a given ecosystem and/or environment condition(s), for example.

As part of the continuous learning and analysis, engineering and technology design alternatives can be integrated to compensate for one component(s)' capability with other component(s) that are already included in the machine built and/or added as part of the mutation to compensate for failure(s) of a given component. An ability to understand the design mitigations that are built-in to the machine 110-112 and/or can be added to the machine 110-112 (e.g., through a software update, new hardware accessory, etc.) increases a likelihood that a component overcompensates when another component of the machine 110-112 enters a failure mode. However, the same capability may also help the machine 110-112 enter a fail-safe mode rather than a catastrophic failure mode for the entire machine 110-112 and/or one or more machine components, for example.

A mutational gene is a gene that compensates for an under-performing feature gene or a sub-optimal performing gene by changing the conditions under which such a gene is performing to rectify the impact of those anomalies in those genes. A performance enhancing machine gene (MuGene) sequence 120-122 combines different strands of a genetic composition in association with the machine 110-112 and its functionality to improve performance given one or more operating conditions, usage variations, tasks, etc.

Specific genes 120-122 can be recognized in each machine 110-112 product family through data analytics, machine/deep learning, etc., and can be correlated with product capability(-ies). A product capability can be formed as a collection of these genes 120-122 coming together to perform a specific operation. For example, an ability of a computed tomography (CT) scanner to scan a patient can be linked to various genetic underpinnings such as radiation dose, high voltage, detector fidelity, reconstruction algorithm(s), stability of gantry, noise avoidance, etc. Certain examples first determine how these genes individually adjust to a changing operating context and, then, collectively compensate to derive an expected outcome utilizing machine learning and collective memory. Mutation and/or other adjustment to the gene sequence 120-122 for the machine 110-112 can be determined based on this analysis.

At block 1250, the evaluation of block 1220 is processed to determine whether an improvement can occur in the MuGene 120-122 and the operating conditions for the machine 110-112. For example, the genetic configuration 120-122 of the machine 110-112 may be sufficient to perform a task and/or otherwise operate in the machine's operating condition(s), but a better machine gene sequence 120-122 may exist to improve machine health, performance, etc. As at block 1240, a performance enhancing machine gene and/or gene sequence 120-122 combines different strands of a genetic composition in association with the machine 110-112 and its functionality to improve performance given one or more operating conditions, usage variations, tasks, etc. One or more genes can be replaced and/or the overall gene sequence 120-122 can be mutated to provide an improved machine gene sequence 120-122 to configure the machine 110-112 for operation, for example.

When an improvement can be made, at block 1260, a mutation and/or replacement gene 120-122 is determined to improve configuration, performance, and/or machine health of the machine 110-112. For example, a gene mutation/enhancement can be incrementally expanded to proactive, preventative, and/or predictive intervention as applicable generic characteristic(s) 120-122 are locked down and solidified for a given ecosystem and/or environment condition(s), for example. As such, a genetic characteristic or a combination of characteristics 120-122 related to the machine or its component (e.g., software, firmware, and/or hardware) 110-112 can be mutated and/or enhanced to improve machine 110-112 configuration, performance, health, etc.

At block 1270, the machine gene sequence 120-122 is set according to the change from block 1240 and/or 1260, if any. For example, the MuGene 120-122 can be adjusted in one or more genes, replaced with another gene sequence, etc., to reconfigure the machine 110-112 and/or machine operation. The machine 110-112 then operates according to the updated MuGene 120-122.

In certain examples, the machine(s) 110-112 and associated MuGene(s) 120-122 (e.g., an imaging device, an imaging workstation, a health information system, etc.), taken individually and/or as a fleet of machines, etc., can be modeled as a digital twin and/or processed according to an artificial neural network and/or other machine/deep learning network model to determine gene mutations, identify and/or predict errors/faults/discrepancies, etc. Using one or more artificial intelligence models, such as a digital twin, neural network model, etc., one or more real-life system can be modeled, monitored, simulated, and prepared for field force automation management.

A digital representation, digital model, digital “twin”, or digital “shadow” is a digital informational construct about a physical system, process, etc. That is, digital information can be implemented as a “twin” of a physical device/system/person/process and information associated with and/or embedded within the physical device/system/process. The digital twin is linked with the physical system through the lifecycle of the physical system. In certain examples, the digital twin includes a physical object in real space, a digital twin of that physical object that exists in a virtual space, and information linking the physical object with its digital twin. The digital twin exists in a virtual space corresponding to a real space and includes a link for data flow from real space to virtual space as well as a link for information flow from virtual space to real space and virtual sub-spaces. For example, the machine(s) 110-112 and associated MuGene(s) 120-122 can be modeled under a variety of operating conditions using digital twin(s). Gene replacement, mutation, etc., can be determined through a digital twin modeling and analysis, for example.

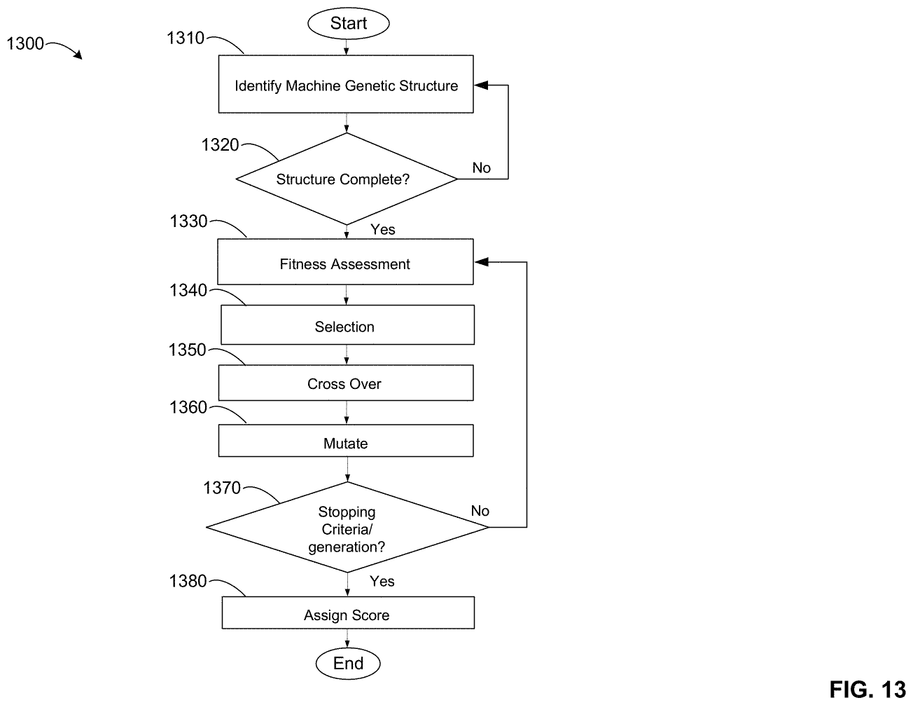

FIG. 13 illustrates a flow diagram of an example method 1300 to analyze and score the genetic structure 120-122 of a machine 110-112. The example method 1300 can be formed from executable program instructions stored in memory and executable by at least one processor to implement the method 1300, for example. At block 1310, the genetic structure 120-122 of the machine 110-112 is identified. Several passes or iterations can be executed to identify the machine's genetic structure 120-122. For example, a first pass can identify and collect composition genetics of the genetic structure 120-122 of the machine 110-112 such as its manufacture, composition, variance against tolerance, software, etc. A second pass, for example, can identify and collect performance genetics of the genetic structure 120-122 of the machine 110-112 such as the performance of gene(s) 120-122 under specific operating conditions. A third pass, for example, can identify and collect health genetics of the genetic structure 120-122 of the machine 110-112 such as the composition and performance to classify health of different output(s) associated with the machine 110-112 (e.g., obtaining an x-ray imaging, performing an ablation, preprocessing raw image data, etc.).

At block 1320, the genetic identification of block 1310 continues until the genetic structure 120-122 of the machine 110-112 has been completely identified. For example, the genetic structure 120-122 is evaluated to determine whether it is in alignment with specific output(s) that the machine 110-112 is designed to provide per customer request. If so, then, at block 1330, a fitness assessment of the genetic structure 120-122 of the machine 110-112 for desired output(s) is assessed. For example, a rank is determined and assigned for each output based on composition genetics (e.g., hardware, software, and/or firmware) and health genetics for the sequence 120-122.

At block 1340, a best performing system configuration is selected at different performance conditions based on the composition genetics, performance genetics, and health genetics of the machine's gene sequence 120-122 to drive machine health and performance while optimizing composition, for example. At block 1350, best performing genetic structure(s) are identified and stacked in cross-over based on the composition genetics, performance genetics, and health genetics to form a genetic code 120-122 for optimal, improved, or otherwise beneficial performance.

At block 1360, mutational capabilities of the genetic code 120-122 are derived based on the fitness assessment of block 1330, selection criteria of block 1340, and cross-over condition of block 1350 to determine one or more mutations. For example, one mutation can include a mutation to induce best performance by the machine 110-112, edge device, and cloud at the same time. Another mutation can include how one gene can compensate for another gene in the machine's configuration.

At block 1370, stopping criteria are evaluated. Stopping criteria represent a multi-generational continuum in which each generation is taken at a face value to be combined with a collective score to both improve performance and to identify a compensating mutational gene. Until stopping criteria have occurred and/or are otherwise satisfied, the genetic structure 120-122 of the machine 110-112 is re-assessed at block 1330 to identify possibility(-ies) for further mutation. However, once stopping criteria are satisfied, at block 1380, a score is assigned to the genetic structure 120-122 for the machine 110-112. Thus, gene sequences 120-122 can be scored and saved for use by the same machine 110-112 and/or other machine(s) 110-112 in a fleet based on their associated score indicating best suitability for particular operating condition(s), output(s), etc. In certain examples, a performance optimizing genetic structure 120-122 can be formed to compensate for one or more criteria under duress conditions (e.g., a failure, error, suboptimal performance, etc.), and that mutation can be converted to a regular gene 120-122 for a next generation of machine 110-112, next configuration, etc.

While an example implementation of the example system 100 is illustrated in FIGS. 1-2, one or more of the elements, processes and/or devices illustrated in FIGS. 1-2 can be combined, divided, re-arranged, omitted, eliminated and/or implemented in any other way. Further, the memory 102, machine configuration processor 104, communication interface 106, and/or, more generally, the system 100 of FIGS. 1-2 can be implemented by hardware, software, firmware and/or any combination of hardware, software and/or firmware. Thus, for example, any of the memory 102, machine configuration processor 104, communication interface 106, and/or, more generally, the system 100 of FIGS. 1-2 can be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)) and/or field programmable logic device(s) (FPLD(s)). When reading any of the apparatus or system claims of this patent to cover a purely software implementation, at least one of the memory 102, the machine configuration processor 104, and the communication interface 106 is/are hereby expressly defined to include a non-transitory computer readable storage device or storage disk such as a memory, a digital versatile disk (DVD), a compact disk (CD), a Blu-ray disk, etc. including the software and/or firmware. Further still, the example system 100 of FIGS. 1-2 can include one or more elements, processes and/or devices in addition to, or instead of, those illustrated in FIGS. 1-2, and/or can include more than one of any or all of the illustrated elements, processes and devices. As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

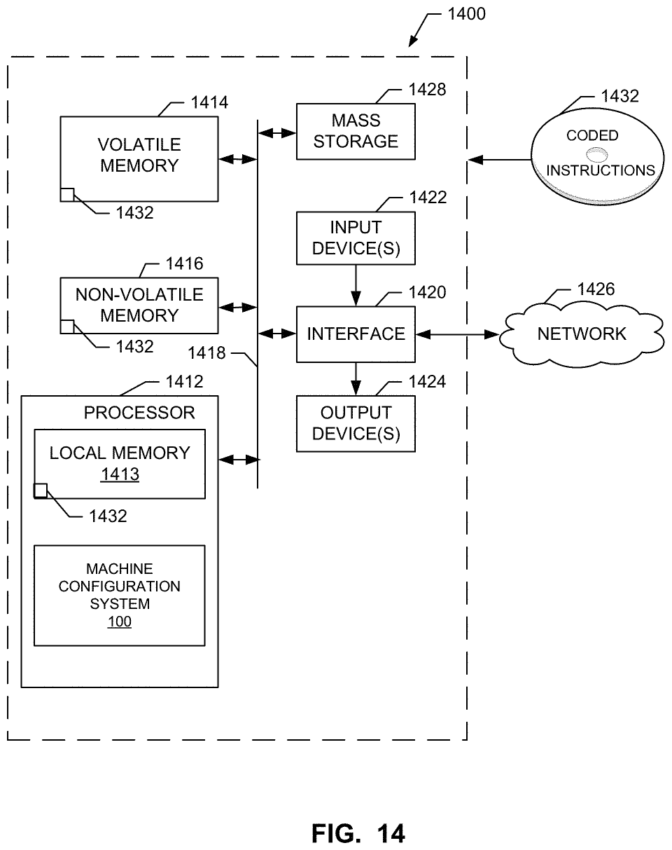

Flowcharts representative of example hardware logic, machine readable instructions, hardware implemented state machines, and/or any combination thereof for implementing the example system 100 of FIGS. 1-2 are shown in FIGS. 12-13. The machine readable instructions can be an executable program or portion of an executable program for execution by a computer processor such as the processor 1412 shown in the processor platform 1400 discussed below in connection with FIG. 14. The program can be embodied in software stored on a non-transitory computer readable storage medium such as a CD-ROM, a floppy disk, a hard drive, a DVD, a Blu-ray disk, or a memory associated with the processor 1412, but the entire program and/or parts thereof could alternatively be executed by a device other than the processor 1412 and/or embodied in firmware or dedicated hardware. Further, although the example program is described with reference to the flowcharts illustrated in FIGS. 12-13, many other methods of implementing the example system 100 can alternatively be used. For example, the order of execution of the blocks can be changed, and/or some of the blocks described can be changed, eliminated, or combined. Additionally or alternatively, any or all of the blocks can be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

As mentioned above, the example processes of FIGS. 12-13 can be implemented using executable instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc. can be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, and (7) A with B and with C. As used herein in the context of describing structures, components, items, objects and/or things, the phrase “at least one of A and B” is intended to refer to implementations including any of (1) at least one A, (2) at least one B, and (3) at least one A and at least one B. Similarly, as used herein in the context of describing structures, components, items, objects and/or things, the phrase “at least one of A or B” is intended to refer to implementations including any of (1) at least one A, (2) at least one B, and (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, and (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, and (3) at least one A and at least one B.

FIG. 14 is a block diagram of a processor platform 1400 structured to execute the instructions of FIGS. 12 and/or 13 to implement the example medical machine configuration system 100 of FIGS. 1-2. The processor platform 1400 can be, for example, a server, a personal computer, a workstation, a self-learning machine (e.g., a neural network), an Internet appliance, and/or any other type of computing device.

The processor platform 1400 of the illustrated example includes a processor 1412. The processor 1412 of the illustrated example is hardware. For example, the processor 1412 can be implemented by one or more integrated circuits, logic circuits, microprocessors, GPUs, DSPs, or controllers from any desired family or manufacturer. The hardware processor can be a semiconductor based (e.g., silicon based) device. In this example, the processor 1412 implements the example system 100 and its components as shown in FIGS. 1 and/or 2.

The processor 1412 of the illustrated example includes a local memory 1413 (e.g., a cache). The processor 1412 of the illustrated example is in communication with a main memory including a volatile memory 1414 and a non-volatile memory 1416 via a bus 1418. The volatile memory 1414 can be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 1416 can be implemented by flash memory and/or any other desired type of memory device. Access to the main memory 1414, 1416 is controlled by a memory controller. The memory 102 can be implemented using one or more of the memory 1413, 1414, 1416.

The processor platform 1400 of the illustrated example also includes an interface circuit 1420 (e.g., the communication interface 106). The interface circuit 1420 can be implemented by any type of interface standard, such as an Ethernet interface, a universal serial bus (USB), a Bluetooth® interface, a near field communication (NFC) interface, and/or a PCI express interface.

In the illustrated example, one or more input devices 1422 are connected to the interface circuit 1420. The input device(s) 1422 permit(s) a user to enter data and/or commands into the processor 1412. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 1424 are also connected to the interface circuit 1420 of the illustrated example. The output devices 1424 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuit 1420 of the illustrated example, thus, typically includes a graphics driver card, a graphics driver chip and/or a graphics driver processor.

The interface circuit 1420 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 1426. The communication can be via, for example, an Ethernet connection, a tech subscriber line (DSL) connection, a telephone line connection, a coaxial cable system, a satellite system, a line-of-site wireless system, a cellular telephone system, etc.

The processor platform 1400 of the illustrated example also includes one or more mass storage devices 1428 for storing software and/or data. Examples of such mass storage devices 1428 include floppy disk drives, hard drive disks, compact disk drives, Blu-ray disk drives, redundant array of independent disks (RAID) systems, and digital versatile disk (DVD) drives.

The machine executable instructions 1432 of FIGS. 12 and/or 13 can be stored in the mass storage device 1428, in the volatile memory 1414, in the non-volatile memory 1416, and/or on a removable non-transitory computer readable storage medium such as a CD or DVD.

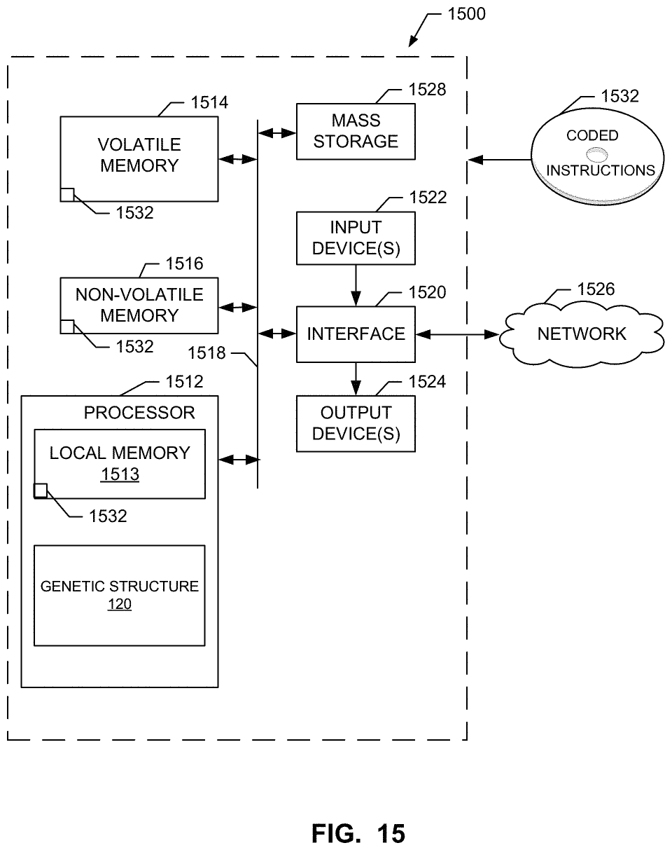

FIG. 15 is a block diagram of a processor platform 1500 structured to execute the instructions of FIGS. 12 and/or 13 as part of the machine 110-112 to implement the example MuGene 120-122 of FIGS. 1-2. The processor platform 1500 can be, for example, a server, a personal computer, a workstation, a self-learning machine (e.g., a neural network), an Internet appliance, and/or any other type of computing device.

The processor platform 1500 of the illustrated example includes a processor 1512. The processor 1512 of the illustrated example is hardware. For example, the processor 1512 can be implemented by one or more integrated circuits, logic circuits, microprocessors, GPUs, DSPs, or controllers from any desired family or manufacturer. The hardware processor can be a semiconductor based (e.g., silicon based) device. In this example, the processor 1512 can form part of the example machine 110-112 and its components as shown in FIGS. 1 and/or 2 including the MuGene 120-122.

The processor 1512 of the illustrated example includes a local memory 1513 (e.g., a cache). The processor 1512 of the illustrated example is in communication with a main memory including a volatile memory 1514 and a non-volatile memory 1516 via a bus 1518. The volatile memory 1514 can be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 1516 can be implemented by flash memory and/or any other desired type of memory device. Access to the main memory 1514, 1516 is controlled by a memory controller.

The processor platform 1500 of the illustrated example also includes an interface circuit 1520. The interface circuit 1520 can be implemented by any type of interface standard, such as an Ethernet interface, a universal serial bus (USB), a Bluetooth® interface, a near field communication (NFC) interface, and/or a PCI express interface.

In the illustrated example, one or more input devices 1522 are connected to the interface circuit 1520. The input device(s) 1522 permit(s) a user to enter data and/or commands into the processor 1512. The input device(s) can be implemented by, for example, an audio sensor, a microphone, a camera (still or video), a keyboard, a button, a mouse, a touchscreen, a track-pad, a trackball, isopoint, and/or a voice recognition system.

One or more output devices 1524 are also connected to the interface circuit 1520 of the illustrated example. The output devices 1524 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuit 1520 of the illustrated example, thus, typically includes a graphics driver card, a graphics driver chip and/or a graphics driver processor.

The interface circuit 1520 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 1526. The communication can be via, for example, an Ethernet connection, a tech subscriber line (DSL) connection, a telephone line connection, a coaxial cable system, a satellite system, a line-of-site wireless system, a cellular telephone system, etc.

The processor platform 1500 of the illustrated example also includes one or more mass storage devices 1528 for storing software and/or data. Examples of such mass storage devices 1528 include floppy disk drives, hard drive disks, compact disk drives, Blu-ray disk drives, redundant array of independent disks (RAID) systems, and digital versatile disk (DVD) drives.

The machine executable instructions 1532 of FIGS. 12 and/or 13 can be stored in the mass storage device 1528, in the volatile memory 1514, in the non-volatile memory 1516, and/or on a removable non-transitory computer readable storage medium such as a CD or DVD.

From the foregoing, it will be appreciated that example methods, apparatus and articles of manufacture have been disclosed that provide new, technologically advanced medical machine configuration, maintenance, monitoring, and repair. The disclosed methods, apparatus and articles of manufacture provide a technological improvement through representing machine configuration and control as a genetic sequence that can be mutated, modified, stored, relayed, etc., and also improve the efficiency of using a computing device by transforming the computing device into a genetic sequencer for diagnosis, repair, and other configuration of connected medical systems. The disclosed methods, apparatus and articles of manufacture are accordingly directed to one or more improvement(s) in the functioning of a computer. Machine genetic sequences or structures can drive automatic, dynamic adjustments/mutations, both proactive and reactive, between machines and/or between machines and a coordinator system without manual human intervention.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

What is claimed is:

1. An apparatus comprising:
memory including instructions for execution by at least one processor and a machine genetic structure specifying composition, performance, and health of a machine; and
at least one processor to execute the instructions to at least:
evaluate the machine genetic structure with respect to an operating condition of the machine to identify at least one of a discrepancy or an opportunity for improvement for the machine genetic structure to satisfy the operating condition;
determine a mutation of the machine genetic structure from a first sequence to a second sequence to address the at least one of a discrepancy or an opportunity for improvement to satisfy the operating condition; and
set the machine genetic structure from the first sequence to the mutation of the second sequence to configure the machine for operation according to the machine genetic structure.

2. The apparatus of claim 1, wherein the at least one processor includes:
a gene analyzer to analyze the machine genetic structure;
a gene modifier to mutate the machine genetic structure; and
a gene communicator to transmit the machine genetic structure.

3. The apparatus of claim 1, wherein the operating condition includes at least one of a) a task to be executed by the machine or b) a parameter to configure the machine.

4. The apparatus of claim 1, wherein the discrepancy indicates an error at the machine.

5. The apparatus of claim 1, wherein the at least one processor is to store the mutation to transmit to a second machine.

6. The apparatus of claim 1, wherein the at least one processor is to evaluate the machine genetic structure with respect to an operating condition of the machine to identify at least one of a discrepancy or an opportunity for improvement for the machine genetic structure to satisfy the operating condition by comparing the machine genetic structurer to at least one of a) a set of stored machine genetic structures or b) a plurality of machine genetic structures associated with a fleet including the machine and a plurality of additional machines.

7. The apparatus of claim 1, wherein the machine genetic structure is formed as a function of hardware, software, and operating conditions of the machine leveraging additional machine genetic structures from a cloud-based system via an edge device to configure the machine.

8. The apparatus of claim 1, wherein the machine genetic structure includes a data structure to alter the configuration of the machine, specify the performance of the machine with respect to the operating condition, and establish a boundary for the health of the machine in operating with respect to the operating condition.

9. A non-transitory computer readable storage medium comprising instructions which, when executed, cause a machine to at least:
evaluate a machine genetic structure with respect to an operating condition of the machine to identify at least one of a discrepancy or an opportunity for improvement for the machine genetic structure to satisfy the operating condition, the machine genetic structure specifying composition, performance, and health of the machine;
determine a mutation of the machine genetic structure from a first sequence to a second sequence to address the at least one of a discrepancy or an opportunity for improvement to satisfy the operating condition; and
set the machine genetic structure from the first sequence to the mutation of the second sequence to configure the machine for operation according to the machine genetic structure.

10. The non-transitory computer readable storage medium of claim 9, wherein the operating condition includes at least one of a) a task to be executed by the machine or b) a parameter to configure the machine.

11. The non-transitory computer readable storage medium of claim 9, wherein the discrepancy indicates an error at the machine.

12. The non-transitory computer readable storage medium of claim 9, wherein the instructions, when executed, cause the machine to store the mutation to transmit to a second machine.

13. The non-transitory computer readable storage medium of claim 9, wherein the instructions, when executed, cause the machine to evaluate the machine genetic structure with respect to an operating condition of the machine to identify at least one of a discrepancy or an opportunity for improvement for the machine genetic structure to satisfy the operating condition by comparing the machine genetic structurer to at least one of a) a set of stored machine genetic structures or b) a plurality of machine genetic structures associated with a fleet including the machine and a plurality of additional machines.

14. The non-transitory computer readable storage medium of claim 9, wherein the machine genetic structure is formed as a function of hardware, software, and operating conditions of the machine leveraging additional machine genetic structures from a cloud-based system via an edge device to configure the machine.

15. A method comprising:
evaluating, by executing an instruction using at least one processor, a machine genetic structure with respect to an operating condition of the machine to identify at least one of a discrepancy or an opportunity for improvement for the machine genetic structure to satisfy the operating condition, the machine genetic structure specifying composition, performance, and health of the machine;
determining, by executing an instruction using the at least one processor, a mutation of the machine genetic structure from a first sequence to a second sequence to address the at least one of a discrepancy or an opportunity for improvement to satisfy the operating condition; and
setting, by executing an instruction using the at least one processor, the machine genetic structure from the first sequence to the mutation of the second sequence to configure the machine for operation according to the machine genetic structure.

16. The method of claim 15, wherein the operating condition includes at least one of a) a task to be executed by the machine or b) a parameter to configure the machine.

17. The method of claim 15, wherein the discrepancy indicates an error at the machine.

18. The method of claim 15, further including storing the mutation to transmit to a second machine.

19. The method of claim 15, wherein evaluating the machine genetic structure with respect to an operating condition of the machine to identify at least one of a discrepancy or an opportunity for improvement for the machine genetic structure to satisfy the operating condition further includes comparing the machine genetic structurer to at least one of a) a set of stored machine genetic structures or b) a plurality of machine genetic structures associated with a fleet including the machine and a plurality of additional machines.

20. The method of claim 15, wherein the machine genetic structure is formed as a function of hardware, software, and operating conditions of the machine leveraging additional machine genetic structures from a cloud-based system via an edge device to configure the machine.
